(19) United States
(12) Patent Application Publication
(10) Pub. No.: US 2004/0116931 A1
(43) Pub. Date: Jun. 17, 2004

(54) Vertebrae fixation device and method of use

(76) Inventor: Gregory D. Carlson, Irvine, CA (US)

Correspondence Address: Kit M. Stetina, STETINA BRUNDA GARRED & BRUCKER, 75 Enterprise, Suite 250, Aliso Viejo, CA, 92656, US

(21) Appl. No.: 10/322,011

(22) Filed: Dec. 17, 2002

(51) Int. Cl. A61B 17/56

(52) U.S. Cl. 606/70

(57) ABSTRACT

An apparatus and method for retaining at least two vertebrae of a spinal column in a desired spatial relationship are disclosed. The apparatus includes at least two anchor plates that are configured to be individually attached to at least two individual vertebrae. Each of the anchor plates is individually attached to a vertebra. The anchor plates are aligned prior to attaching each of the individual anchor plates. The anchor plates attached to the individual vertebrae from a frame structure. A connection member is attached to the frame structure.

### CROSS-REFERENCE TO RELATED APPLICATIONS

(Not Applicable)

### STATEMENT RE: FEDERALLY SPONSORED RESEARCH/DEVELOPMENT

(Not Applicable)

### BACKGROUND OF THE INVENTION

The present invention relates generally to spinal fixation devices and more particularly to an apparatus and method for retaining vertebrae of a spinal column in a desired spatial relationship.

There are various known devices for retaining two vertebrae of a spinal column in a desired spacial relationship. Such retaining devices include either a rod or a plate connecting the two vertebrae.

The rod or plate is connected to anchors, e.g., using screws. The retaining device (i.e., two anchors connected to each other via a rod or plate) is then attached to the desired vertebrae of the spinal column. Such a device may be inserted and attached through tubes or a minimally invasive surgical procedure. However, it is difficult to accurately position and attach the device in the desired position. An X-ray may be needed to verify that the device is accurately positioned prior to attaching the device to the vertebrae. Furthermore, due to placement difficulties, more than one procedure may be required in order to get the fixation device properly positioned.

Therefore, there is a need for a retaining device that is relatively easy to position and attach to vertebrae in a desired spacial relationship.

### BRIEF SUMMARY OF THE INVENTION

An apparatus and method for retaining at least two vertebrae of a spinal column in a desired spatial relationship are disclosed. The apparatus includes at least two anchor plates that are configured to be individually attached to at least two individual vertebrae. Each of the anchor plates is individually attached to a vertebra. The anchor plates are aligned prior to attaching each of the individual anchor plates to a vertebra. The anchor plates attached to the individual vertebra form a frame structure. A connection member, such as a rod, is attached to the anchor plates forming the frame structure and is used for holding the vertebrae in the desired spatial relationship.

The anchor plates may include one or more fins to increase fixation strength.

An anterior loading bracket may be placed over the connection member and secured to the anchor plate.

Preferably, the anchor plates include a channel (or channels) configured to hold the connection member.

Connection fasteners are used to hold the connection member in place such that a desired range of movement is possible. The connection fastener may be a set screw.

Frictional locking sleeves may be included to allow dynamism (slight movement).

The anchor plates are sufficiently smaller than the vertebrae to facilitate positioning of an anchor plate on a vertebra.

The method includes: attaching a first anchor plate to a first one of the vertebrae and attaching a second anchor plate to a second one of the vertebrae to form a frame structure. The first and second anchor plates are then connected using a connection member. The connection member is secured to the anchor plates so that a desired range of movement is possible.

There may be more than two anchor plates connecting more than two vertebrae.

### BRIEF DESCRIPTION OF THE DRAWINGS

These as well as other features of the present invention will become more apparent upon reference to the drawings wherein:

FIG. 1 is an elevational view of a first embodiment of a vertebrae fixation device that uses a rod to connect anchor plates that are first individually attached to at least two vertebrae and further includes an anterior loading bracket to secure the rod in place;

FIG. 2 is a cross sectional view taken generally along line 2 of FIG. 1;

FIG. 3 is a cross sectional view taken generally along line 8 of FIG. 1;

FIG. 4 is a cross sectional view taken generally along line 4 of FIG. 2;

FIG. 5 is an exploded view of the first embodiment of the vertebrae fixation device shown in FIG. 1;

FIG. 6 is an elevational view of a second embodiment of a vertebrae fixation device that uses a rod to connect anchor plates that are first individually attached to at least two vertebrae;

FIG. 7 is a cross sectional view taken generally along line 7 of FIG. 6;

FIG. 8 is a cross sectional view taken generally along line 8 of FIG. 6;

FIG. 9 is a cross sectional view taken generally along line 9 of FIG. 6; and

FIG. 10 is an exploded view of the embodiment of the second vertebrae fixation device shown in FIG. 6.

### DETAILED DESCRIPTION OF THE INVENTION

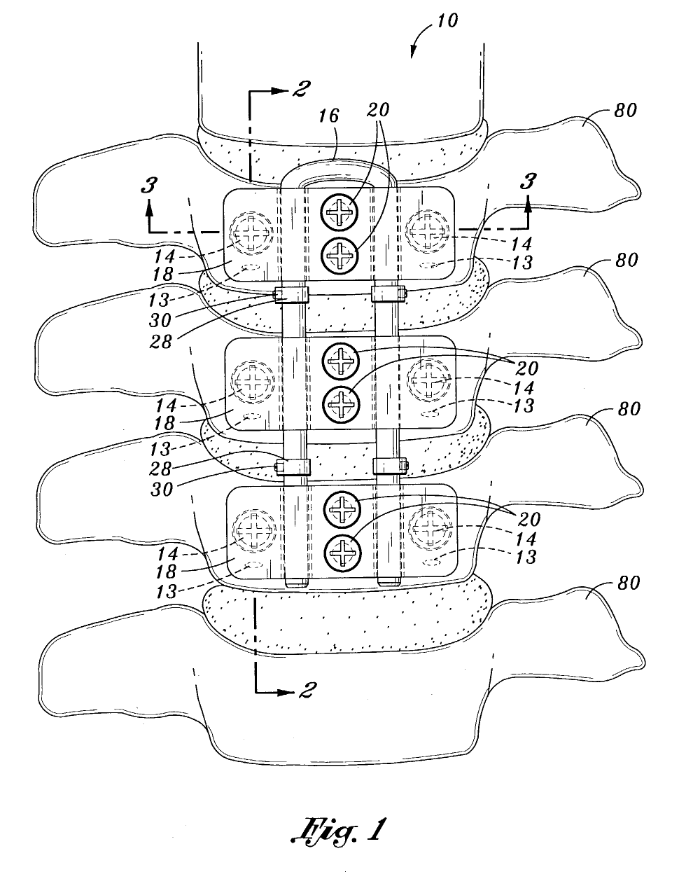

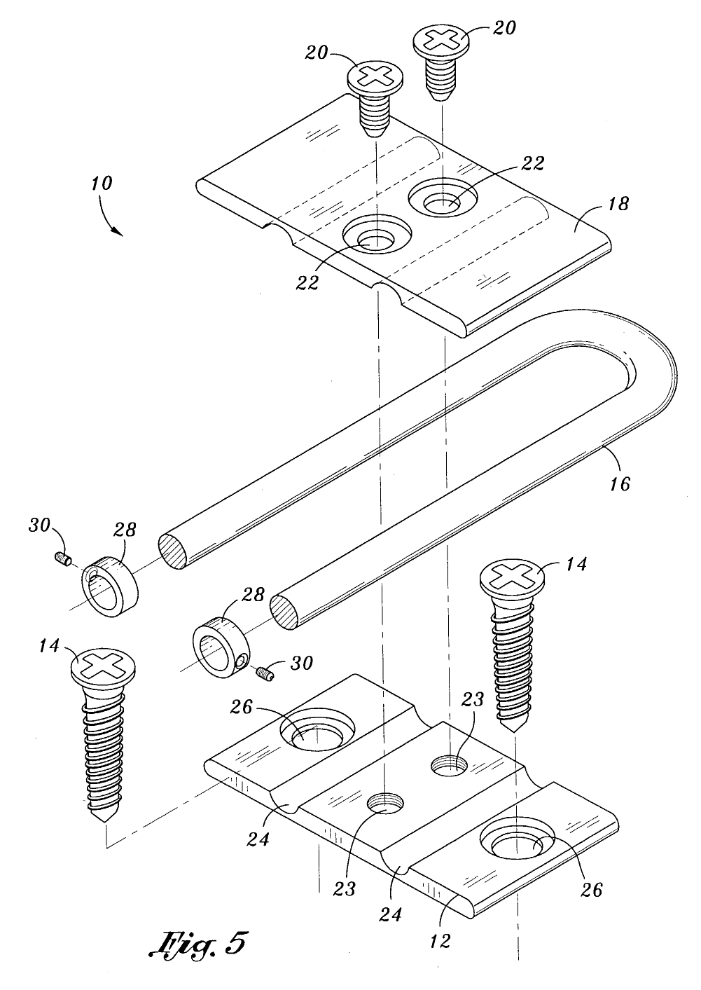

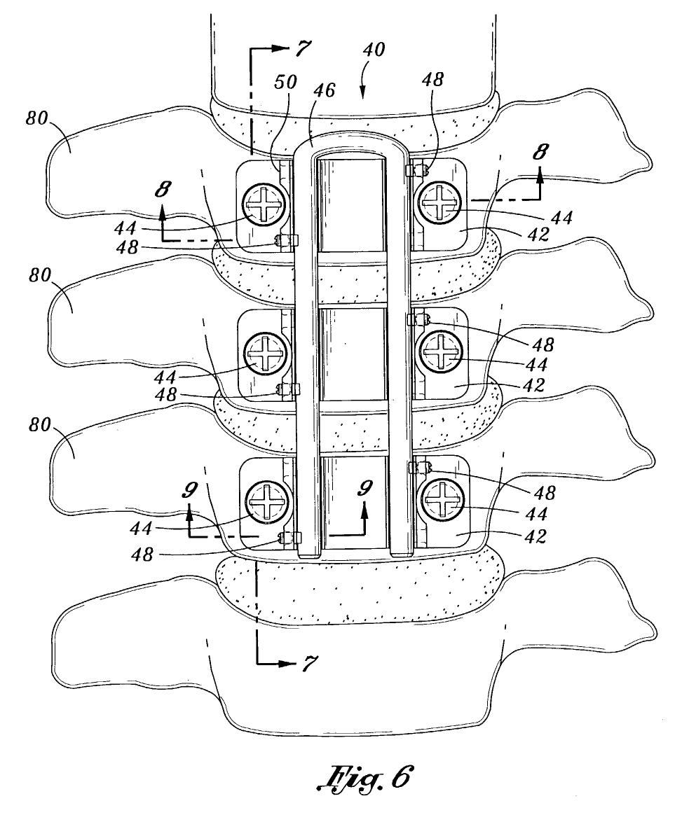

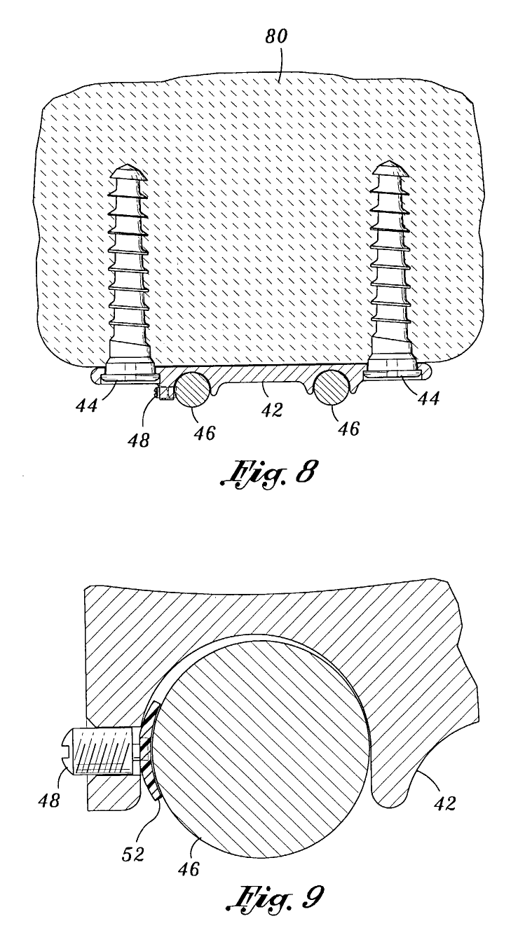

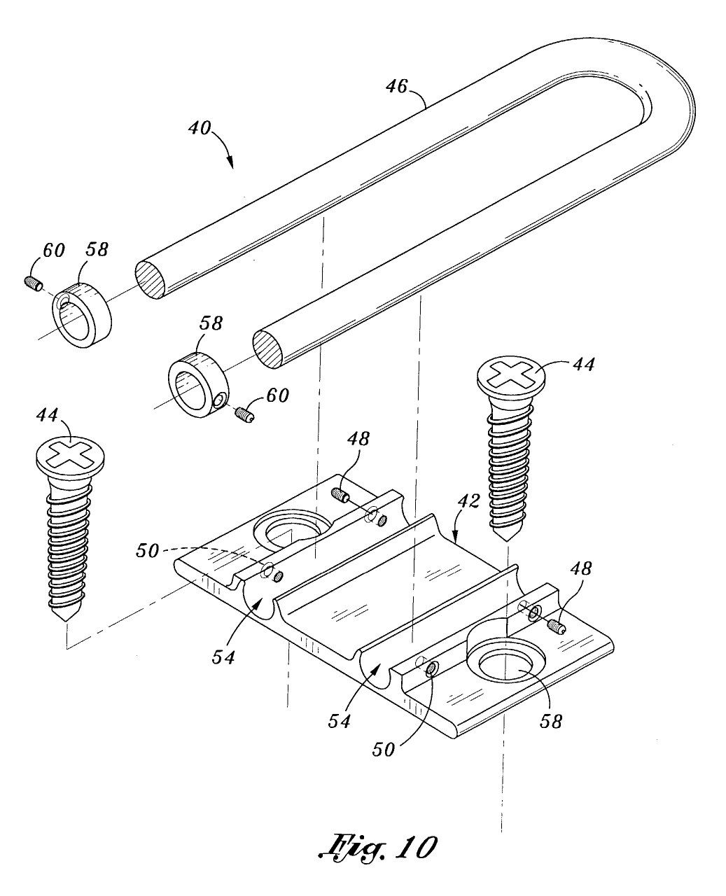

Referring now to the drawings wherein the showings are for purposes of illustrating preferred embodiments of the present invention only, and not for purposes of limiting the same, FIGS. 1-5 illustrate a first embodiment of a vertebrae fixation device that uses a rod or other connection member to connect foot plates (known herein as anchor plates) that are first individually attached to at least two vertebrae. This first embodiment includes an anterior loading bracket to secure the connection member to the anchor plates that are first individually attached to the vertebrae. FIGS. 6-10 illustrate a second embodiment of the vertebrae fixation device which is similar to the first embodiment but does not include the anterior loading bracket.

Referring to the first embodiment shown in FIGS. 1-5, the vertebrae fixation device 10 attaches to two or more vertebrae 80 of a spinal column. In the embodiment shown, the vertebrae fixation device 10 is attached to three vertebrae 80. It will be appreciated that the device 10 can be attached to different numbers of vertebrae, e.g., two, four, etc.

The device 10 includes anchor plates 12 that are individually attached to the vertebrae 80. A frame structure (that is attached to the vertebrae) is formed by the anchor plates 12. This individual implantation of the anchor plates allows for individual control of the vertebral bodies.

Preferably, but optionally, the anchor plate 12 may include one or more fins 13 to increase fixation strength. In the embodiment shown, one anchor plate 12 is attached to an individual vertebrae 80 using screws 14. The screws 14 are inserted through holes 26 in the anchor plate 12. In the embodiment shown, two screws 14 are used to attach each anchor plate 12 to a vertebrae 80. The anchor plates 12 can be inserted and attached using a minimally invasive procedure. The anchor plates 12 are small enough that they can be positioned in proper alignment (with the other anchor plate(s)) on the vertebrae 80.

A frame structure is formed by each of the individually attached anchor plates 12. Individually attaching the anchor plates 12 to form a frame structure and then connecting the frame structure with a connection member, such as a rod 16, allows for relatively easy and accurate positioning of the fixation device 10. This also prevents the potential need for X-rays and/or multiple procedures which are typically associated with vertebrae fixation devices. The anchor plates 12 may be implanted before or after disectomy (neural decompression). The anchor plates 12 may be used as distraction points between vertebral bodies to increase visualization of neural decompression.

Once all of the anchor plates 12 have been aligned and secured to the vertebrae 80, a connection member 16, is connected to the frame structure. The connection member 16 may be any configuration to include varying shapes or material to allow for a stabilizing connector between anchor plates 12. For example, the connection member 16 may be a rod. As shown in the illustrated embodiments, the connection member 16 may be a U-shaped rod. In other embodiments, the connection member 16 may have a different configuration, for example, the connection member may be a plate. Importantly, the anchors (frame structure) are first implanted. The connection member 16 is then positioned within and connected to the frame structure. The connection member 16 is used to connect the individual anchor plates 12. After intervertebral reconstruction, e.g., with graft, cage or strut material, vertebral anchors may be compressed at the time the anchor plates 12 are attached with the connection member 16.

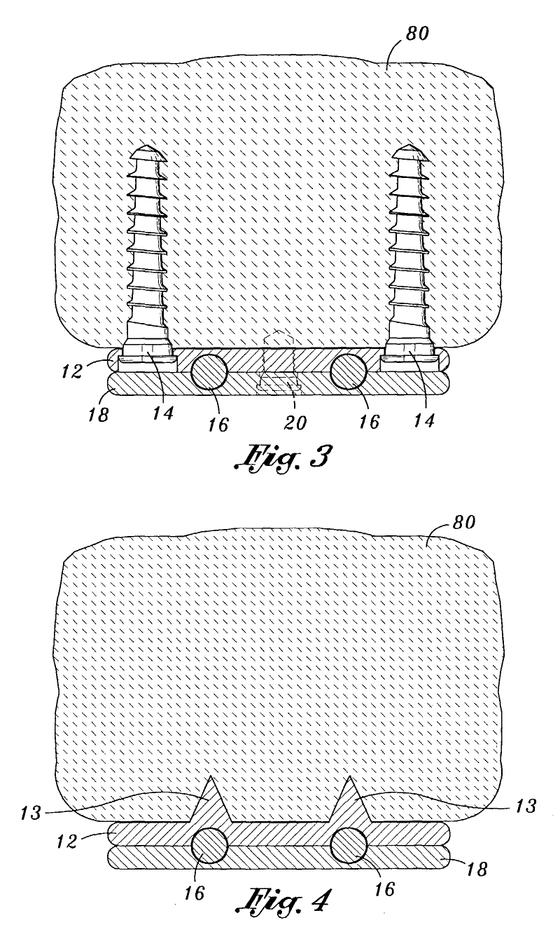

As best seen in FIGS. 3-5, the connection member 16 is preferably inserted in a channel 24. In the embodiment shown in FIGS. 1-5, a U-shaped rod 16 is used. Therefore, the anchor plates 12 include two channels 24 spaced at a sufficient distance that each of the legs of the U-shaped rod 16 rests within one of the channels 24.

After the connection member 16 has been positioned on the anchor plates 12, anterior loading brackets 18 are positioned on top of the anchor plates 12. The anterior loading bracket 18 holds the connection member 16 in place so that the vertebrae 80 are held in the desired spatial relationship. Once positioned, the anterior loading bracket 18 is secured in place, for example, using screws 20. The screws 20 are placed through screw holes 22 in the anterior loading bracket 18 and threaded screw holes 13 in the anchor plate 12.

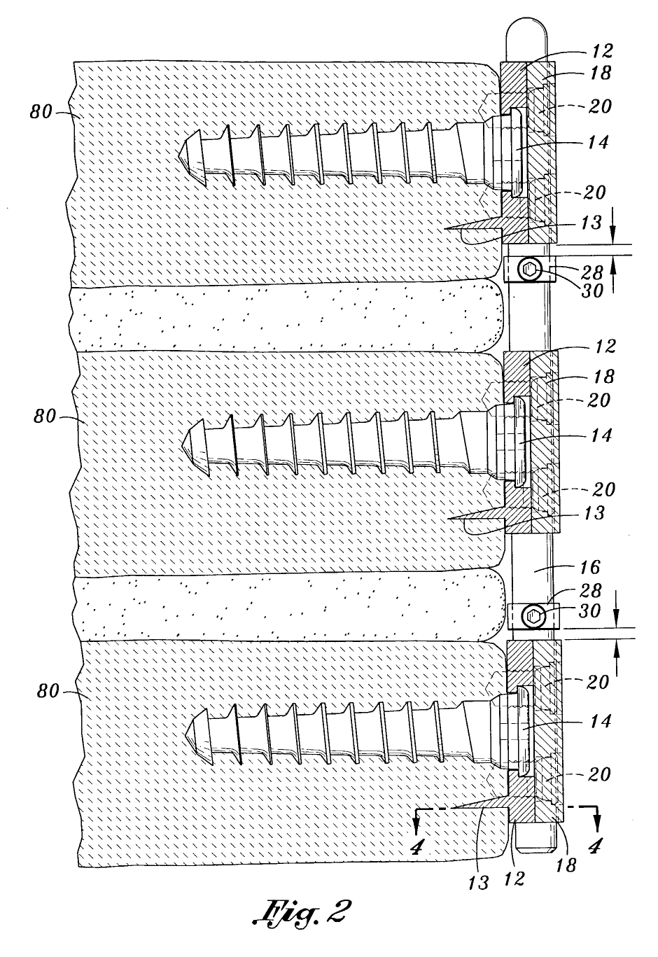

Dynamic compression or settling occurs between the anchor plates 12 and the connection member 16 by means of low friction connecting linkage. Static settings will be allowed through high friction or rigid connecting points. Locking sleeves 18, such as those shown in FIG. 2) may be positioned at varying points along the connection member 16 distal to the anterior locking bracket 18 to quantitatively control the amount of settling or slippage, i.e., to allow for dynamism. The locking sleeves 18 may be held in place using set screws 20.

The vertebrae fixation device 10 may be constructed of bioresorbable material or other materials as deemed necessary to provide intervertebral stability during healing.

In exemplary embodiments, the vertebrae fixation device 10 is a low profile construct.

FIGS. 1-5 illustrate one embodiment. It will be appreciated that other embodiments are possible. For example, the connection member may be a plate instead of a rod. The anchor plate may not include fins or the anchor plate may include multiple fins. Locking sleeves 28 may be omitted or may be positioned in a manner that causes the device 10 to keep the vertebrae 80 in a static position.

Another possible embodiment, shown in FIGS. 6-10 does not include anterior loading brackets 18 or fins on the anchor plates. Like the embodiment shown in FIGS. 1-5, the embodiment shown in FIGS. 6-10 includes anchor plates 42 that are individually attached to the vertebrae 80, for example using screws 44 that are inserted through holes 56 in the anchor plate 42.

As with the embodiment shown in FIGS. 1-5, after the anchor plates 42 are positioned and attached to the vertebrae 80, a connection member 46, such as a rod, is positioned to connect the anchor plates 42 (which form the frame structure) as shown in FIG. 6. As best shown in FIG. 10, a channel 54 is configured to hold the connection member 46 in position.

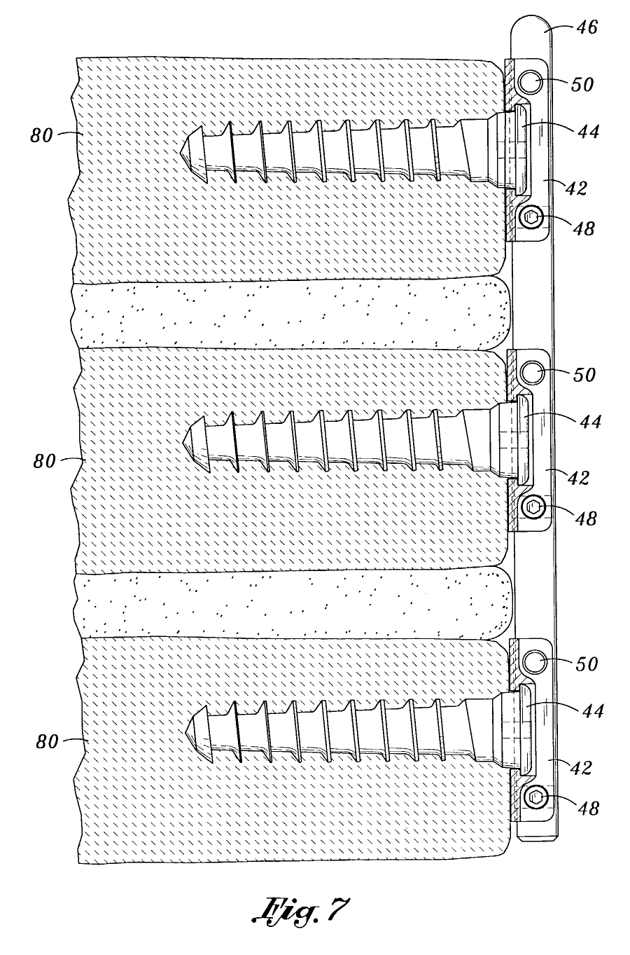

Locking sleeves 58, such as those shown in FIG. 7) may be positioned at varying points along the rod 46 distal to the anterior locking bracket 48 to quantitatively control the amount of settling or slippage, i.e., to allow for dynamism. The locking sleeves 58 may be held in place using set screws 60.

While an illustrative and presently preferred embodiment of the invention has been described in detail herein, it is to be understood that the inventive concepts may be otherwise variously embodied and employed and that the appended claims are intended to be construed to include such variations except insofar as limited by the prior art.

What is claimed is:

1. An apparatus for retaining at least two vertebrae of a spinal column in a desired spatial relationship, the apparatus comprising:
at least two anchor plates configured to be individually attached to at least two respective vertebrae;
a plurality of fasteners, wherein at least one fastener is used to individually attach each of the anchor plates to the respective vertebrae, and wherein a frame structure is formed by the anchor plates when the anchor plates have been individually attached to the respective vertebrae; and
a connection member configured to be attached to at least two of the anchor plates of the frame structure that has been formed by individually attaching the anchor plates to the vertebrae.

2. The apparatus of claim 1, wherein a respective anchor plate comprises at least one fin configured to be inserted into a respective vertebra.

3. The apparatus of claim 1, further comprising an anterior loading bracket that is placed over the connection member and fastened to the anchor plate.

4. The apparatus of claim 1, further comprising connection fasteners configured to hold the connection member in place such that the desired spatial relationship is maintained.

5. The apparatus of claim 4, wherein the connection fasteners are set screws.

6. The apparatus of claim 1, further comprising frictional locking sleeves that provide for a limited range of motion while still maintaining the desired spatial relationship.

7. The apparatus of claim 1, wherein the anchor plate is sufficiently smaller than the vertebra to facilitate positioning of the anchor plate on the vertebra.

8. The apparatus of claim 1, wherein the connection member is a rod.

9. The apparatus of claim 1, wherein the anchor plates each comprises a channel configured to hold the connection member.

10. A method for retaining at least two vertebrae of a spinal column in a desired spatial relationship, the method comprising:
(a) attaching a first anchor plate to a first one of the vertebrae;
(b) attaching a second anchor plate to a second one of the vertebrae, wherein the first and second anchor plates form a frame structure;
(c) connecting the first and second anchor plates of the frame structure using a connection member; and
(d) securing the connection member to the frame structure so that the desired spatial relationship is maintained.

11. The method of claim 10, wherein a respective anchor plate comprises at least one fin configured to be inserted into a respective vertebra.

12. The method of claim 10, wherein the connection member is secured to each of the anchor plates using an anterior loading bracket.

13. The method of claim 10, wherein there are more than two anchor plates connecting more than two vertebrae and attaching each of the anchor plates is attached to a separate vertebra and all of the anchor plates form the frame structure.

14. The method of claim 10, wherein the connection member is secured to each of the anchor plates using at least one set screw per anchor plate.

15. The method of claim 10, further comprising positioning locking sleeves on the connection member to allow for a limited range of movement while still maintaining the desired spatial relationship.

16. The method of claim 10, wherein the connection member is a rod.

17. The method of claim 10, wherein connecting the first and second anchor plates using a connection member comprises connecting the first and second anchor plates by placing the connection member in a channel in each of the anchor plates.
